(19) United States
(12) Patent Application Publication
(10) Pub. No.: US 2014/0207110 A1
(43) Pub. Date: Jul. 24, 2014

(54) VASCULAR ACCESS DEVICE FOR REPRODUCIBLE INSERTION OF A CANNULA INTO A PUNCTURE SITE

(71) Applicant: Fresenius Medical Care Deutschland GmbH, Bad Homburg (DE)

(72) Inventor: Joerg JONAS, Bogota (CO)

(73) Assignee: Fresenius Medical Care Deutschland GmbH, Bad Homburg (DE)

(21) Appl. No.: 14/160,912

(22) Filed: Jan. 22, 2014

Related U.S. Application Data
(60) Provisional application No. 61/755,535, filed on Jan. 23, 2013.

(30) Foreign Application Priority Data
Jan. 23, 2013 (DE) 102013001105.1

(51) Int. Cl. A61B 17/34

(52) U.S. Cl. CPC A61B 17/3403
USPC 604/510; 604/272; 604/179

(57) ABSTRACT

A vascular access device for reproducible insertion of a cannula, comprising a carrier, a holder and a guide device, such that the guide device is designed as an inclined plane on one side and serves to support and guide a cannula, so that accurate guidance of the cannula is ensured, always in the same puncture angle and at the same depth of penetration in a puncture channel.

The present invention relates to a vascular access device which allows a reproducible insertion of a cannula into a puncture site.

In treating critically or chronically ill patients, it may happen within the context of a treatment that infusions, transfusions, blood sampling or blood replacement methods must be performed or administered very frequently. This means that a vascular access must be created each time for such treatment methods. This is perceived by the patient as being unpleasant and painful. Insertion of a new vascular access each time is a very time-consuming procedure for the medical. personnel. To improve the patient's comfort and to simplify the working routines for the medical personnel, various options are available for creating a vascular access for medium- to long-term use.

Thus, for example, central venous catheters (CVC) are used routinely. However, these have the disadvantage that, among other things, they must be cleaned and/or replaced at regular intervals, and infections or venous inflammation, i.e., phlebitis, may occur repeatedly in patients, despite the care taken in caring for the CVC.

In the field of blood purification methods, such as those used in apheresis and in particular in hemodialysis, a good and reliable vascular access is of fundamental importance for an adequate and effective treatment. Therefore to be able to perform regular hemodialysis treatments with a relatively high frequency (several times a week) over a long period of time, special vascular accesses are created, permitting simple and frequent puncturing of the blood vessel for connection to an extracorporeal circulation. Of the various options for vascular access in hemodialysis, native arterio-venous fistulas (AVF) which are formed from an arterial vessel and a venous vessel have the best long-term results. The vein dilates within a short period of time under the pressure of the arterial bloodstream. Therefore, a blood flow high enough for an efficient dialysis treatment is achieved in the fistula area and then a puncture area of sufficient size is formed. In addition, the blood vessel develops a more stable vascular wall, which tolerates frequent puncturing.

A native arterio-venous fistula has a natural biocompatibility because it consists of endogenous tissue. In contrast with central venous catheters, the risk of infections and inflammation of the vein is therefore rare with a native arterio-venous fistula because the puncture site usually heals completely and is hermetically sealed by a wound scab after the cannula has been removed.

However, the AVF in chronic hemodialysis with more than 300 punctures per year using cannulas that are sharp and have a large lumen is exposed to very high stresses. If these punctures are always performed in the same area of the AVF, this may be perceived as less painful by the patient, but the so-called areal puncture leads to rapid wear and tear on the AVF because of irreparable tissue damage as a result of dilatation up to and including the development of aneurysms, stenoses, infections, peripheral ischemias and ultimately the loss of the AVF.

Therefore, from the standpoint of vascular surgery, the “rope-ladder method” is preferred, in which new puncture sites over the length of the AVF are used each time. The choice of puncture sites and the punctures are performed primarily by specially trained dialysis personnel. However, the puncture of new puncture sites each time with this method causes a severe puncture pain for the patient. Sequelae here again may also include the tissue damage mentioned above.

Another improved puncture method is the “buttonhole puncture technique.” In this technique, the same puncture channel, puncture angle and cannula diameter are always used for each puncture. For the first six to twelve punctures, which are usually performed within two weeks to one month, a sharply cutting cannula, puncture needle or the like is used in each case. Over this period of time, a puncture channel is formed from connective tissue, which assumes the function of a guiderail for subsequent punctures. After the puncture channel has been formed, the additional punctures are made with a blunt cannula having a rounded tip in order to prevent damage to the puncture channel and the AVF. The puncture channel heals outwardly with a small wound scab, which must be removed before each renewed puncture. The puncture heals inwardly in the blood vessel and thus seals it tightly. Repeated puncture at the same puncture site causes scar tissue to form in the puncture channel, and there is also permanent damage to the local nerve tissue. As in the case of pierced ears, the puncture channel no longer grows back together. A stable puncture channel, which permits repeated, painless and fistula-protecting puncture along the puncture channel, then develops within a few weeks.

Although the buttonhole technique is advantageous, its practical implementation is difficult. The puncture procedure requires a great deal of experience, sensitivity and a high precision in guiding the needle, because in order to form a puncture channel, the first punctures must be made with a sharply cutting cannula in precisely the same puncture channel and angle. Punctures of the same puncture site in each case can therefore be performed only by specially trained personnel and should always be performed by the same person if possible.

Because of the puncture problems, the use of this method has been very limited so far and has been restricted primarily to a specially trained group of people. In home dialysis in particular, where this method would be a great advantage from the standpoint of increased frequency of treatment and future self-puncture, but when trained personnel are not present on site, the puncture problems constitute the main obstacle for use of this technique.

U.S. Pat. No. 4,883,053 describes a device for insertion of catheters and needles for biopsy, wherein an angular dimension can be adjusted for insertion of the catheters and/or needles.

The application WO2012/178109 as well as the corresponding application WO 2012/088458 of the same patent family describe a needle guide assembly including a needle guide body with multiple guide channels, wherein each guide channel is able to house a needle with a specific needle size respectively a specific insertion angle. The needle guide body is attached to an image producing device, wherein the inspection of a subcutaneous target is determined by an ultrasound probe.

The object of the present invention is now to supply vascular access devices, which allow a reproducible insertion of a cannula to simplify the performance of the buttonhole technique.

In addition, the vascular access device according to the invention should increase safety in creating a buttonhole for the patient by avoiding faulty punctures due to inaccurate guidance of the needle. In addition, the vascular access device should offer the possibility of measuring and/or limiting the depth of penetration of the cannula into a lumen, in particular a blood vessel.

Another object of the invention is to make available the buttonhole technique with the vascular access device according to the present invention to an expanded group of users, in particular patients in home dialysis. This object is achieved by the subject matter of Claim 1. A vascular access device for reproducible insertion of a cannula into a puncture site is used here, allowing secure and accurate positioning, support and guidance of a cannula. The vascular access device comprises a carrier, a holder and a guide device for adjusting an angle such that the guide device has an inclined plane.

This object is also achieved by a method of using the vascular access device according to the invention especially for the reproducible insertion of a cannula as recited in Claim 11.

Additional advantageous embodiments of the invention are derived from the dependent claims.

The term “cannula” as used here comprises all types of hollow or solid needles that are suitable for producing a puncture channel. These include in particular transfusion needles, puncture needles, dialysis needles, infusion needles and injection needles, but also catheters and the like.

The vascular access device according to the invention is described below in greater detail with reference to the drawings.

FIG. 1 shows schematically a top view of a vascular access device with a carrier, a holder, a guide device and a cannula.

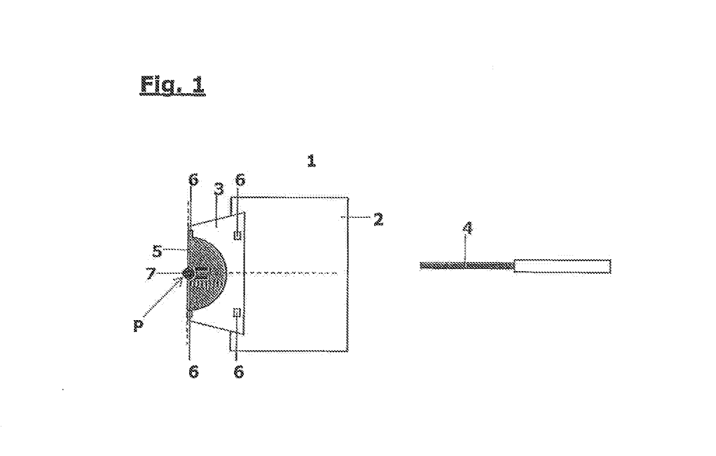

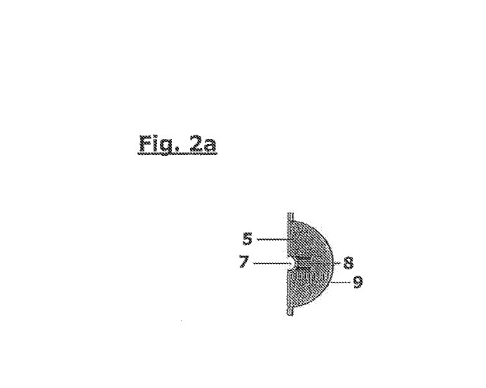

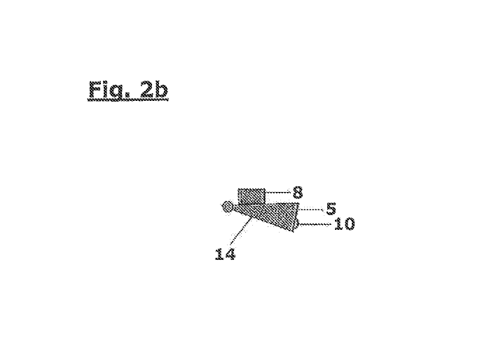

FIGS. 2a and 2b show a guide device from various perspectives.

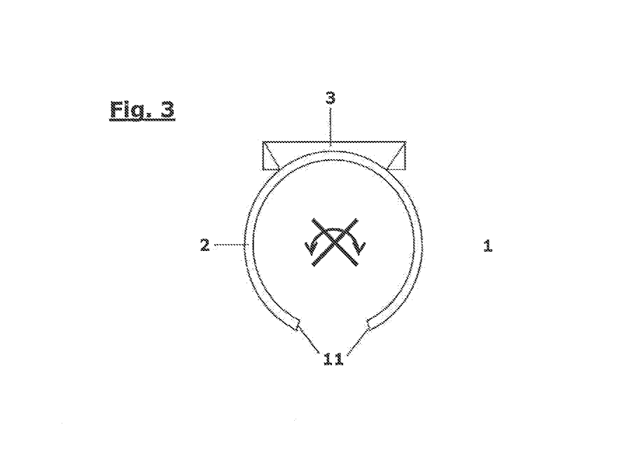

FIG. 3 shows schematically a front view of a vascular access device having a carrier and a holder.

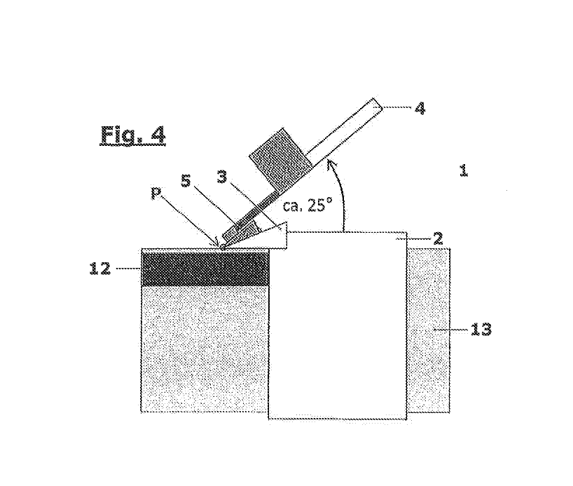

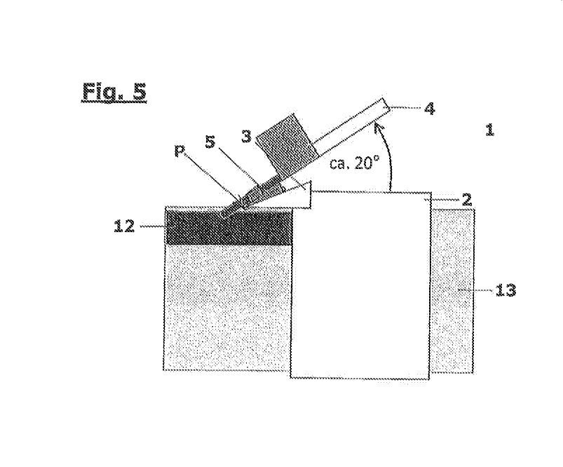

FIGS. 4 and 5 show schematically a sectional diagram of a vascular access device having a cannula in different positions.

FIG. 1 shows a vascular access device (1) in a view from above. The vascular access device (1) for reproducible insertion of a cannula (4) comprises a carrier (2), a holder (3) and a guide device (5). The guide device (5) is characterized on the one hand by an inclined plane (14). The inclined plane (14) serves to support the cannula (4) and allows targeted guidance of the cannula (4) in a puncture channel. Using this guide device (5), which is inserted into the holder (3) of the carrier (2), various angular positions can be defined with respect to a planar surface, preferably the surface of the skin above an AVF. The position of a lumen, in particular a blood vessel, is of great importance in the selection of an optimal puncture angle. Especially in the puncture of blood vessels, their position must be determined accurately in each case because the position may vary greatly from one patient to the next. Therefore, the puncture will be performed at a steeper angle when the lumen is deeper, and the puncture will be performed at a shallower angle when the lumen is superficial. The differentiated angular position offers the advantage that the puncture angle can be adjusted accurately to each individual patient. In the case of the vascular access device (1) according to the invention, the puncture angles may be selected from a range of 5° to 45°, preferably of 10° to 30°, especially preferably of 20° to 25°. The initial puncture into the vessel is performed at a larger angle in order to keep the lumen of the puncture opening in the AVF as small as possible, and then the cannula (4) is advanced at an angle that is smaller by approximately 0.5° to 10°. The specific embodiment of the guide device supports the user in finding the ideal puncture angle as well as the ideal angle of advance.

The guide device (5) may be designed as a semicircular disk, such that the semicircular disk has a half-round half-round recess (7) which is preferably arranged centrally on the straight edge. With the help of the half-round recess (7), which is aligned with the side of the vascular access device (1) facing the puncture site (P), the puncture site (P) can be targeted accurately. The holder (3) may advantageously also have a semicircular recess to receive the guide device (5).

For securely connecting the guide device (5) to the holder (3), they are equipped with connecting elements (6) in the form of clip connections, snap connections or hook connections. The connecting elements (6) may consist of receiving elements and locking elements. The holder (3) therefore has receiving elements, preferably on its distal end, which may be designed as grooves, notches, slots, recesses, etc. On its distal lateral ends, the guide device (5) preferably has locking elements by means of which the guide device (5) can be connected to the holder (3). The locking elements are pins, hooks, buttons or the like, which may be introduced into the receiving elements of the holder (3). The receiving elements as well as the locking elements may also be mounted on the holder (3) and/or on the guide device (5) in the opposite manner.

The guide device (5) may be mounted rotatably in the holder (3) within the angular range indicated above by means of an axle on the distal end of the holder (3).

FIG. 2a shows a guide device (5), again in a view from above. A needle guide (8) for secure fastening and guidance of a cannula (4), which is advantageously situated above the half-round recess (7), is mounted on the surface of the guide device (5). A scale (9) or gradation, in particular a length gradation, is applied next to the needle guide (8). The scale (9) or gradation serves to measure the forward advance of the cannula (4), so that a reproducible depth of penetration can be ensured with each puncture. However, the needle guide (8) may also be designed, so that it has an end stop by means of which the forward advance of the cannula (4) can be limited to thus ensure protection from puncturing a lumen (12), in particular a blood vessel.

The needle guide (8), shown in sectional view in FIG. 2b, may be designed in the form of a rail, a channel, a holder or the like, into which the cannula (4) can be inserted, clamped or attached.

FIG. 2b shows an inclined plane (14) on one side of the guide device (5). This yields the puncture axis in three-dimensional space, as indicated in FIG. 1, through the alignment of the carrier (2) parallel to the longitudinal axis of a body or extremity, preferably an arm, and through the inclined plane (14) of the guide device (5), which is mounted in the holder (3) of the carrier (2).

The design of the guide device (5) with an inclined plane (14) offers the advantage that the guide device (5) can be applied to a body flatly and thus forms a stable base. The guide device (5) is thus secured well over the entire available angle range to prevent unintentional slippage or shifting of a preset puncture angle.

The inclined plane (14) also offers the advantage that the puncture angle can be adjusted in a variable manner according to the position of the lumen to be punctured.

The guide device (5) can also be positioned quickly, reliably and easily in the holder (3) of the carrier (2) of the vascular access device (1) by means of the inclined plane (14).

Various angular positions can be set accurately by means of a locking mechanism (10). The locking mechanism (10) is situated between the holder (3) and the guide device (5) and is embodied in the form of a locking catch, for example. The locking catch is preferably mounted between the guide device (5) and the holder (3) on the guide device (5) on the longitudinal access of the vascular access device (1). On the opposite side, there are recesses (not shown) in the holder (3) with which the locking catch can engage at predetermined angular positions. The locking force of the catch mechanism (10) can be overcome by slight vertical pressure on the guide device (5), and the guide device (5) can be shifted into another angular position. The rotational path is preferably limited by an end stop to prevent complete rotation of the guide device (5). A desired puncture angle can be set with the help of the catch mechanism (10) for reproducible insertion of a cannula (4) into a puncture channel.

FIG. 3 shows a front view of a vascular access device (1) comprising a carrier (2) and a holder (3). The carrier (2) may be designed as a strip or a band in the form of a ring. The carrier (2) is preferably in the form of an armband or bracelet and should encircle a patient's body, preferably an extremity, in particular an arm in a slip-proof and twist-proof manner. The diameter of the carrier (2) should be adjustable in a variable manner, so that it can be adapted to the individual patient. The variability of the diameter can be implemented by connecting the two ends (11) of the carrier using a clasp, a Velcro-type fastener or a hinge or by connecting the carrier ends (11) into one another or over one other or by similar methods. When applying the vascular access device (1), care must be taken to ensure that the diameter selected is not too small, so that neither the pressure exerted by the carrier (2) on the AVF nor the blood flow prevailing therein is impaired.

As already shown in FIG. 1, the holder (3) is attached to one side of the carrier (2). The holder (3) and the carrier (2) may be designed as a one-piece or two-piece element. In the case of a two-piece embodiment, both the carrier (2) and the holder (3) will have connecting elements (6), for example, clip connections, snap connections or hook connections, so that the two elements can be joined to one another. The connecting elements (6) may be embodied as receiving elements in the form of grooves, notches, slots, recesses, etc. on one side (2) of the carrier, while the proximal end of the holder (3) has connecting elements as locking elements in the form of pins, hooks, buttons, etc. for connecting the carrier (2) to the holder (3). The receiving and locking elements may of course also be mounted on the carrier (2) and/or the holder (3) in the reverse order. The carrier (2) and the holder (3) may preferably be made of a flexible material that is gentle to the skin, for example, plastic, leather, non-stretching fabric bands or the like.

In one embodiment, the carrier (2) and the holder (3) form a unit to receive the guide device (5), which can be inserted into the holder (3). The ring-shaped carrier (2) in the form of an armband or bracelet has a sufficient width of 5 cm to 15 cm and thus forms a stable base for a precise alignment and fixation of the guide device (5).

In an alternative embodiment, a vascular access device (1) comprising a carrier (2), a holder (3) and a guide device (5) may be manufactured in one piece—as a unit. The entire vascular access device (1) must be sterilized because the vascular access device (1), in particular the guide device (5), is used in the immediate vicinity of a previously disinfected but open puncture site (P).

In an alternative embodiment, because of these hygienic requirements, the vascular access device (1) may preferably be composed of two units—the carrier (2) including the holder (3) as the first unit and the guide device (5) as the second unit—such that as the first unit, either the carrier (2) and the holder (3) may form one unit or they may be manufactured as two elements. The guide device (5) in this case may be designed as a sterile disposable article. Such a design offers the advantage that only the part of the vascular access device (1) which must necessarily be attached in the vicinity of the puncture site (P) for accurate alignment and guidance of the cannula (4) must be sterile. This means that, as a sterile disposable article, the guide device (5) may be inserted into the holder (3) attached to the carrier prior to use and then discarded after use. The larger and much more cost-intensive unit (the carrier (2) and the holder (3)) of the vascular access device (1)) may be reused after being disinfected or sterilized.

In another alternative embodiment, the vascular access device (1) may be composed of the carrier (2) as the first unit and the holder (3) with the function device (5) as the second unit. In this combination, the sterility of the holder (3) and the guide device (5) would have to be ensured.

One method for reproducible insertion of a cannula by means of a vascular access device (1) will now be explained on the basis of an example, in which the vascular access device (1) comprises a carrier (2), a holder (3) and a guide device (5), a cannula which is inserted into, clamped or attached onto the needle guide (8) of the guide device (5), a first puncture angle is set by means of the locking device (10), the cannula (4) is advanced along the needle guide (8) into a puncture channel, until reaching a first depth of penetration, a second puncture angle is set by means of the guide device (5) and the cannula (4) is advanced up to a second depth of penetration.

As shown in FIG. 4, a vascular access device (1) is secured on a body (13), preferably on an extremity, especially preferably on a patient's arm. After inserting a guide device (5) into a holder (3) of a carrier (2), a cannula (4) can be inserted into, clamped on or attached to a needle guide (8). A cannula (4) may optionally be first inserted into a guide device (5) and then placed in a holder (3) of the carrier (2).

In an alternative embodiment, the cannula (4) may already be premounted on the guide device (5) and may be supplied as a disposable article set.

A first puncture angle may be set in the range of 5° to 45° by means of a locking mechanism (10) as described above. For example, a puncture angle of 25° to 35° to the opening of the puncture site (P) may be selected and the cannula (4) (sharp or blunt) may be inserted along a puncture channel. The advance and thus a first depth of penetration of the cannula (4) may be read on a scale (9) on the top side of the guide device (5) and recorded as needed. The first depth of penetration is reached when the vascular wall has been punctured by the cutting edge of the cannula (4), which corresponds to a depth of penetration of 5 mm to 10 mm, depending on the position of the AVF. As soon as the vascular wall of the AVF has been penetrated, there is a return flow of fluid into the cannula (4) when using a hollow needle and the advance is stopped briefly. The further advance into a lumen (12), in particular a blood vessel, may then be performed at a reduced second angle as shown in FIG. 5. Through slight vertical pressure on the guide device 5, the locking catch is released and can be locked again to the holder (3) at a reduced second angle of 10° to 24°, for example. In this position, the cannula (4) is then advanced in the lumen (12), preferably in the blood vessel up to a second depth of penetration. Here again, the advance of the cannula (4) in the lumen (12) is determined on the basis of the scale (9) on the function device (5) and is recorded for the next punctures, so that in addition to a reproducible puncture angle, a reproducible depth of penetration can also be ensured. With the second depth of penetration, the cannula (4) is advanced in the lumen (12) by an additional 10 to 17 mm, preferably by 13 to 17 mm. In this specific exemplary embodiment, a needle length of 25 mm is assumed, with the total depth of penetration amounting to max. 23 mm. To avoid puncturing the lumen (12) and/or the blood vessel, the needle guide (8) has an end stop beyond which the cannula (4) cannot be advanced further.

After the successful puncture, the cannula (4) is released from the guide device (5), and the vascular access device (1) is repositioned for another puncture or is removed completely.

Alternatively, the guide device (5) may also be released from the holder (3) first, and then the carrier (2) and the holder (3) may be removed. Following this, the guide device (5) is removed from the cannula (4).

After removing the vascular access device (1), the cannula (4) is reliably secured on the patient in accordance with the relevant operating instructions.

The vascular access device (1) is provided primarily for supporting the medical personnel and/or for patients for self-puncture for the period of time until a puncture channel has been completely formed. During the formation of the puncture channel, the puncture is always performed using sharp cannulas (4), so the vascular access device (1) offers the advantage that secure insertion of the cannula (4) into the same puncture site (P) in each case and along the spatial axis, which is always the same, can be guaranteed by an accurately adjustable puncture angle and an accurately definable depth of penetration, and the variance in the deviation from this axis by the medical personnel or the self-puncturing patient can be minimized. However, the vascular access device (1) may also be used further after forming the stable puncture channel. Since the puncture of a stable puncture channel is then performed only using a blunt cannula, this also reduces the risk of injury to the user, so the buttonhole technique may also be used easily and safely by means of the vascular access device (1) according to the invention, permitting use directly by the patient in the field of home dialysis or by people without medical training after brief instruction. This in turn also contributes toward improved comfort for the patient.

1. A vascular access device (1) for reproducible insertion of a cannula (4) comprising a carrier (2), a holder (3) and a guide device (5), wherein the guide device (5) is embodied as an inclined plane (14) on one side.

2. The vascular access device (1) according to claim 1, characterized in that the inclined plane (14) forms an angle of 5° to 45°, preferably 10° to 30°, especially preferably 20° to 25° to a planar surface.

3. The vascular access device (1) according to claim 1 or 2, characterized in that the guide device (5) is designed as a semicircular disk, such that the semicircular disk has a half-round recess (7) on the straight cut edge, preferably disposed centrally.

4. The vascular access device (1) according to claim 1 to 3, characterized in that the guide device (5) is mounted to rotate about an axis on the distal end of the holder (3).

5. The vascular access device (1) according to claim 1, characterized in that the guide device has a needle guide (8), and a scale or a gradation (9) is provided next to the needle guide (8).

6. The vascular access device (1) according to claim 1, characterized in that a locking mechanism (10) in the form of a locking catch is attached between the holder (3) and the guide device (5).

7. The vascular access device (1) according to claim 1, characterized in that the carrier (2) is designed as a band or strip in a ring shape, preferably on the form of an armband or a bracelet.

8. The vascular access device (1) according to claim 7, characterized in that the diameter of the carrier (2) can be adjusted variably by means of snap closures, Velcro-type fasteners or hinges or by intermeshing or stacked carrier ends (4).

9. The vascular access device (1) according to claim 1, characterized in that the carrier (2) and the holder (3) are each designed in one part or in two parts, and the guide device (5) is inserted into the holder (3).

10. The vascular access device (1) according to claim 9, characterized in that the vascular access device (1) is assembled from
a unit comprising a carrier (2), a holder (3) and a guide device (5) or
a first unit comprising a carrier (2) and the holder (3) and a second unit comprising a guide device (5) or
a first unit comprising a carrier (2) and a second unit comprising a holder (3) and a guide device (5).

11. A method for reproducible insertion of a cannula (4) by means of a vascular access device (I), characterized in that into the vascular access device (1) comprising a carrier (2), a holder (3) and a guide device (5)
a cannula (4) is inserted into or clamped on or attached to the needle guide (8) of the guide device (5),
a first puncture angle is set by means of the locking device (10),
the cannula (4) is advanced along the needle guide (8) into a puncture channel until reaching a first depth of penetration,
a second puncture angle is adjusted by means of the guide device (5), and
the cannula (4) is advanced to a second depth of penetration.

12. The method for reproducible insertion of a cannula (4) by means of a vascular access device (1) according to claim 11, characterized in that
the first puncture angle may be selected from a range of 25° to 35° and
the second puncture angle may be selected from a range of 10° to 24°.

13. The method for reproducible insertion of a cannula (4) by means of a vascular access device (1) according to claim 12, characterized in that
the first depth of penetration is between 5 mm and 10 mm, and
the second depth of penetration is between 10 mm and 17 mm, preferably between 13 and 17 mm.

14. The method for reproducible insertion of a cannula (4) by means of a vascular access device (1) according to claim 11 or 12, characterized in that the angular positions are set by means of a locking mechanism (10), which is mounted between the holder (3) and the guide device (5).

15. The method according to claim 11 with a vascular access device (1) according to claim 1.
